(19) United States
(12) Patent Application Publication
(10) Pub. No.: US 2010/0239539 A1
(43) Pub. Date: Sep. 23, 2010

(54) Methods for promoting differentiation and differentiation efficiency

(76) Inventors: George L. Sing, New York, NY (US); Vivienne S. Marshall, San Antonio, TX (US)

Correspondence Address: LINDA O. PALLADINO, 45 HONEYSUCKLE COURT, STORMVILLE, NY, 12582, US

(21) Appl. No.: 12/661,640

(22) Filed: Mar. 22, 2010

Related U.S. Application Data
(60) Provisional application No. 61/210,846, filed on Mar. 23, 2009.

(51) Int. Cl. A61K 35/14; C12N 5/078; A61P 7/00

(52) U.S. Cl. 424/93.7; 435/377; 435/372

(57) ABSTRACT

The invention is directed to methods for promoting differentiation of stem cells to hematopoietic cell lineages. The invention is further directed to increasing the differentiation efficiency of hematopoietic stem/progenitor cells. Such methods utilize novel compositions, including but not limited to, Amnion-derived Multipotent Progenitor cells (herein referred to as AMP cells) and conditioned media derived therefrom (herein referred to as Amnion-derived Cellular Cytokine Solution or ACCS), each alone or in combination with each other or other agents.

### CROSS-REFERENCE TO RELATED APPLICATIONS

This application claims priority under 35 USC §119(e) of U.S. Provisional Application No. 61/210,846, filed Mar. 23, 2009, the entirety of which is incorporated herein by reference.

### FIELD OF THE INVENTION

The field of the invention is directed to methods for promoting differentiation of stem cells to hematopoietic cell lineages. The field of the invention is further directed to increasing the differentiation efficiency of hematopoietic stem/progenitor cells. Such methods utilize novel compositions, including but not limited to Amnion-derived Multipotent Progenitor cells (herein referred to as AMP cells) and conditioned media derived therefrom (herein referred to as Amnion-derived Cellular Cytokine Solution or ACCS), each alone or in combination with each other or other agents.

### DESCRIPTION OF RELATED ART

Published U.S. Patent Application No. 20060182724 discloses a method of increasing the growth of stem cells with a growth medium that has been conditioned by an incubation with placental tissue. This method increases the expansion of the stem cell population without substantially inducing differentiation. Miyamoto, K., et al., 2004, Stem Cells 22:433-40) report that human placenta feeder layers support undifferentiated growth of primate embryonic stem cells.

### BACKGROUND OF THE INVENTION

Hematopoietic stem cells (HSC) are stem cells and the early precursor cells which give rise to all the blood cell types including the myeloid lineages (monocytes, macrophages, neutrophils, basophils, eosinophils, erythrocytes, megakaryocytes/platelets and some dendritic cells) and lymphoid lineages (T-cells, B-cells, NK-cells and some dendritic cells). HSCs are found in the bone marrow tissue of certain adult bones (i.e. femur, hip, rib, sternum). Most of these cell types are short-lived and must be replaced every few hours, days, weeks, or months.

The process by which hematopoietic stem cells differentiate to form the many different mature blood cells is called hematopoiesis. It involves the formation of precursors for each of the different kinds of blood cells from the hematopoietic stem cell. The differentiation process occurs principally in the bone marrow, spleen, thymus, and lymph nodes, and is controlled by a complex system of cytokine signals that attempts to maintain an appropriate balance among all of these different types of cells.

Many preclinical and clinical settings require large amounts of hematopoietic cells. For example, patients who have received chemotherapy and/or radiation therapy to deplete their diseased bone marrow require hematopoietic tissue for bone marrow transplant. Patients who have lost large volumes of blood due to injury and/or surgery often require many units of transfused blood to survive. Other patients require individual blood components (i.e. platelets) which need to be extracted from donor blood using a procedure called apheresis. Of particular significance is the loss of blood experienced by soldiers in combat whose injury generally occurs in the field where large inventories of blood and blood components are difficult to store. In addition, many disease states and infections dramatically affect hematopoiesis, resulting in depletion of certain types of blood cells. For example, HIV infection often causes anemia (red blood cell deficiency), neutropenia (neutrophil deficiency), or thrombocytopenia (platelet deficiency), or various combinations of these states, including pancytopenia, which is a deficiency of all different types of blood cells.

Currently, all blood, blood components and bone marrow inventories depend on donations from healthy volunteers. Unfortunately, need far exceeds supply, especially in large cities where traumatic injuries are commonplace, and on the battlefield. Much research has focused on developing artificial blood substitutes, but to date no universally suitable product exists. Thus, it would be extremely useful if one could take stem cells of any type and, by treating them appropriately, cause them to differentiate down a hematopoietic cell lineage pathway. Furthermore, it would also be useful to treat the stem cells such that the differentiation potential of the stem cell population being treated was increased so that a large percentage of the cells in the population are differentiatable. This would result in a significant increase in the availability of hematopoietic lineage cells suitable for transplantation and transfusion and reduce the need to rely exclusively on donated blood tissue. Applicant believes the instant invention described herein provides such methods and compositions, thus fulfilling this unmet medical need.

### BRIEF SUMMARY OF THE INVENTION

It is an object of the instant invention to provide novel methods for differentiation of stem cells into hematopoietic cell lineages. It is further an object of the invention to increase the differentiation efficiency of such stem cell populations. Such methods utilize novel compositions including, but not limited to, Amnion-derived Multipotent Progenitor cells (herein referred to as AMP cells), conditioned media derived therefrom (herein referred to as Amnion-derived Cellular Cytokine Solution or ACCS), and cell products derived therefrom, each alone and/or in combination with each other and/or with other agents including active and/or inactive agents. In a certain particular embodiment, the AMP cells are pooled AMP cells and the ACCS is pooled ACCS.

Accordingly, a first aspect of the invention is a method for differentiating stem cells into hematopoietic lineage cells comprising culturing the stem cells with Amnion-derived Multipotent Progenitor (AMP) cells or Amnion-derived Cellular Cytokine Solution (ACCS). In a particular embodiment the hematopoietic lineage cells are myeloid and/or lymphoid lineage cells.

A second aspect of the invention is a method for increasing the differentiation efficiency of stem cells in a population comprising culturing the stem cells with Amnion-derived Multipotent Progenitor (AMP) cells or Amnion-derived Cellular Cytokine Solution (ACCS).

In specific embodiments of aspects one or two the AMP cells are a feeder layer in the culture or the AMP cells are co-cultured with the stem cells in the culture.

Another specific embodiment of aspects one or two further optionally comprises adding ACCS as a supplement to the stem cell culture.

Still another specific embodiment of aspects one or two is one in which the stem cells are totipotent, pluripotent or multipotent stem cells. In a particular embodiment, the stem cells are embryonic stem cells, fetal stem cells, extraembryonic stem cells or adult stem cells. In a very specific embodiment the adult stem cells are hematopoietic stem cells or hematopoietic progenitor cells. In another embodiment the stem cells are cultured with ACCS prior to differentiation and in still another embodiment the stem cells are cultured with ACCS during differentiation.

A third aspect of the invention is a composition comprising hematopoietic lineage cells which is made by the method of aspect one of the invention. In a particular embodiment the composition of aspect three is one in which the hematopoietic lineage cells are myeloid lineage cells. And in another embodiment of aspect three the hematopoietic lineage cells are lymphoid lineage cells. A further embodiment of aspect three is one in which the composition is a pharmaceutical composition.

A fourth aspect of the invention is a method of treating a disease or disorder of the hematopoietic system of a patient in need thereof comprising administering to the patient a therapeutic amount of the composition of the third aspect of the invention. In particular embodiments of this aspect of the invention the disease or disorder in the hematopoietic system is the result of radiation, chemotherapy, infection, trauma or disease.

A fifth aspect of the invention is a kit comprising the pharmaceutical composition of aspect three.

Other features and advantages of the invention will be apparent from the accompanying description, examples and the claims. The contents of all references, pending patent applications and published patents, cited throughout this application are hereby expressly incorporated by reference. In case of conflict, the present specification, including definitions, will control.

### DEFINITIONS

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of a cell or cell population. The protein marker may be located on the plasma membrane of a cell or in some cases may be a secreted protein.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “hematopoietic stem cell” or “HSC” means a stem cell that is capable of differentiating into both myeloid lineages (i.e. monocytes, macrophages, neutrophils, basophils, eosinophils, erythrocytes, megakaryocytes/platelets and some dendritic cells) and lymphoid lineages (i.e. T-cells, B-cells, NK-cells, and some dendritic cells).

As used herein, the term “Amnion-derived Multipotent Progenitor cell” or “AMP cell” means a specific population of cells selected from the amnion epithelial cells which are derived from the amnion. AMP cells have the following characteristics. They secrete the cytokines VEGF, Angiogenin, PDGF and TGFβ2 and the MMP inhibitors TIMP-1 and TIMP-2. The physiological range of the cytokine or cytokines in the unique combination is as follows: ˜5-16 ng/mL for VEGF, ˜3.5-4.5 ng/mL for Angiogenin, ˜100-165 pg/mL for PDGF, ˜2.5-2.7 ng/mL for TGFβ2, ˜0.68 μg mL for TIMP-1 and ˜1.04 μg/mL for TIMP-2. In addition, AMP cells have not been cultured in the presence of any non-human animal-derived substances, making them and cell products derived from them suitable for human clinical use because they are not xeno-contaminated. In a preferred embodiment, AMP cells are grown in human serum or human serum albumin. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are selected, do not react with antibodies to the stem/progenitor cell markers c-kit (CD117) and Thy-1 (CD90). AMP cells lack c-kit expression as well, although Thy-1 expression increases as the cells are cultured. Finally, AMP cells are not immortal. Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells. AMP cells have previously been described as “amnion-derived cells” (see U.S. Provisional Application Nos. 60/666,949, 60/699,257, 60/742,067, U.S. Provisional Application Nos. 60/813,759, U.S. application Ser. No. 11/392,892, U.S. application Ser. No. 11/724,094, and PCTUS06/011392, each of which is incorporated herein in its entirety).

By the term “animal-free” when referring to certain compositions, growth conditions, culture media, etc. described herein, is meant that no non-human animal-derived substances, such as bovine serum, proteins, lipids, carbohydrates, nucleic acids, vitamins, etc., are used in the preparation, growth, culturing, expansion, storage or formulation of the certain composition or process. By “no non-human animal-derived substances” is meant that the substances have never been in or in contact with a non-human animal body or material so they are not xeno-contaminated. Only clinical grade materials, such as recombinantly produced human proteins and purified human serum albumin, are used in the preparation, growth, culturing, expansion, storage and/or formulation of such compositions and/or processes.

By the term “serum-free” when referring to certain compositions, growth conditions, culture media, etc. described herein, is meant that no non-human animal-derived serum is used in the derivation, preparation, growth, culturing, expansion, storage or formulation of the certain composition or process.

By the term “expanded”, in reference to cell compositions, means that the cell population constitutes a significantly higher yield of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such primary cells are subcultured, each round of subculturing is referred to as a passage. As used herein, “primary culture” means the freshly isolated cell population.

As used herein, the term “differentiation” means the process by which cells become progressively more specialized.

As used herein, the term “differentiation efficiency” means the percentage of cells in a population that are differentiating or are able to differentiate.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein. As used herein, conditioned medium also refers to components, such as proteins, that are recovered and/or purified from conditioned medium or from AMP cells.

As used herein, the term “Amnion-derived Cellular Cytokine Solution” or “ACCS” means conditioned medium that has been derived from AMP cells or expanded AMP cells which have been cultured in a basal medium supplemented with human serum or human serum albumin. Amnion-derived Cellular Cytokine Solution or ACCS has previously been referred to as “amnion-derived cytokine suspension”.

The term “physiological level” as used herein means the level that a substance in a living system is found, for example, in the circulatory system or in a particular microenvironment or biological niche in the living system, and that is relevant to the proper functioning of biochemical and/or biological processes.

create a new composition having more constant or consistent characteristics as compared to the non-pooled compositions. For example, pooled ACCS has more constant or consistent characteristics compared to non-pooled ACCS. Examples of pooled compositions include “SP pools” (more than one ACCS collection/one placenta), “MP1 pools” (one ACCS collection/placenta, multiple placentas), and “MP2 pools” (more than one ACCS collection/placenta, multiple placentas).

The term “therapeutically effective amount” means that amount of a therapeutic agent necessary to achieve a desired physiological effect (i.e. promote hematopoiesis).

The term “lysate” as used herein refers to the composition obtained when cells, for example, AMP cells, are lysed and, optionally, the cellular debris (e.g., cellular membranes) is removed. Lysis may be achieved by mechanical means, by freezing and thawing, by sonication, by use of detergents, such as EDTA, or by enzymatic digestion using, for example, hyaluronidase, dispase, proteases, and nucleases.

The term “cell product” or “cell products” as used herein refers to any and all substances made by and secreted from a cell, including but not limited to, protein factors (i.e. growth factors, differentiation factors, engraftment factors, cytokines, morphogens, proteases (i.e. to promote endogenous cell delamination, protease inhibitors), extracellular matrix components (i.e. fibronectin, etc.).

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

The term “transplantation” as used herein refers to the administration of a composition comprising cells that are either in an undifferentiated, partially differentiated, or fully differentiated form into a human or other animal. Transplantation may also refer to the insertion of a tissue or organ into a subject.

As used herein, the terms “a” or “an” means one or more; at least one.

As used herein, the term “adjunctive” means jointly, together with, in addition to, in conjunction with, and the like.

As used herein, the term “co-administer” or “in combination with” can include simultaneous or sequential administration of two or more agents.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Therapeutic Uses

Blood transfusion—The methods of the invention are suitable for causing differentiation of stem cells down a hematopoietic lineage pathway or increasing the differentiation efficiency of hematopoietic stem and/or progenitor cells 1s, thus creating, for example, a composition comprising blood cells useful for blood transfusion. Such composition may comprise cells of the myeloid lineages or cells of the lymphoid lineage. In a specific embodiment, cells of both the myeloid and lymphoid lineages are present in the composition. In other embodiments, the composition comprises erythrocytes and optionally neutrophils and/or basophils and/or eosinophils and/or megakaryocytes/platelets and/or T-cells and/or B-cells and or NK-cells and/or dendritic cells.

Bone marrow transplantation—The methods of the invention are also suitable for causing differentiation of stem cells down a hematopoietic lineage pathway or increasing the differentiation efficiency of hematopoietic stem and/or progenitor cells, thus creating, for example, a composition comprising blood cells useful for bone marrow transplantation. Such differentiation may occur ex vivo or in vivo.

Uses for Blood Components—The methods of the invention are also suitable for causing differentiation of stem cells down a hematopoietic lineage pathway, thus creating, for example, a composition comprising therapeutically useful blood cell components suitable for transfusion and/or infusion. Such compositions may comprise, for example, platelets, which are cell fragments of the megakaryocyte. Platelets are extremely important in hemostasis (blood clot formation) and are often depleted in patients undergoing chemotherapy and/or radiation treatment. Currently, the only means available for obtaining platelets is apheresis of donated blood, and time-consuming process that relies on healthy volunteer donors.

Treating Hematopoietic Acute Radiation Syndrome—Total body irradiation destroys the hematopoietic stem cells in the bone marrow and reduces the number of adult stem cells in other tissues that are critical for tissue repair and regeneration. Proper bone marrow hematopoietic stem cell repopulation, progenitor cell reconstitution, and mature blood cell production requires a delicate balance between hematopoietic stem cell self-renewal, proliferation, and differentiation. In the bone marrow, a small fraction of hematopoietic stem cells have been found to be radiation resistant and are not killed when the body is irradiated, which is in contrast to the majority of the cycling hematopoietic stem cells which are killed. In order to repopulate the bone marrow, quiescent hematopoietic stem cells are recruited into the proliferative state. Supportive care, such as bone marrow transplantation, has increased survival from hematopoietic ARS, but preventative treatments not involving HLA-matched allogeneic bone marrow stem cell donors are currently not available. Because of the unique properties of AMP cells described herein, Applicants believe that the methods and compositions of the invention are suitable for treating hematopoietic ARS and increasing the differentiation efficiency of hematopoietic stem and/or progenitor cells.

Obtaining and Culturing of Cells

AMP cell compositions are prepared using the steps of a) recovery of the amnion from the placenta, b) dissociation of the cells from the amniotic membrane, c) culturing of the dissociated cells in a basal medium (for example IMDM highly enriched basal medium) with the addition of a naturally derived or recombinantly produced human protein (for example human serum albumin); d) selecting the adherent cells (the AMP cells) and discarding the non-adherent cells from the cell culture, and optionally e) further proliferation of the cells using additional additives and/or growth factors (i.e. human recombinant EGF). Details are contained in US Publication No. 2006-0222634-A1, which is incorporated herein by reference.

Culturing of the AMP cells—The cells are cultured in a basal medium. Such medium includes, but is not limited to, EPILIFE® culture medium for epithelial cells (Cascade Biologicals), OPTI-PROT™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® [symbol] T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology. “Human protein” also is meant to include a human fluid or derivative or preparation thereof, such as human serum or amniotic fluid, which contains human protein. In preferred embodiments, the basal media is STEMLINE® T-cell expansion medium or STEMLINE® II hematopoietic stem cell expansion medium, or OPTI-PRO™ serum-free culture medium, or combinations thereof and the human protein is human albumin at a concentration of at least 0.5% and up to 10%. In particular embodiments, the human albumin concentration is from about 0.5 to about 2%. The human albumin may come from a liquid or a dried (powder) form and includes, but is not limited to, recombinant human albumin, PLASBUMIN® normal human serum albumin and PLASMANATE® human blood fraction (both made by Talecris Biotherapeutics).

In a most preferred embodiment, the cells are cultured using a system that is free of animal products to avoid xeno-contamination. In this embodiment, the culture medium is IMDM highly enriched basal medium, STEMLINE® T-cell expansion medium or STEMLINE® II hematopoietic stem cell expansion medium, OPTI-PROT™ serum-free culture medium, or DMEM culture medium, with human serum albumin (PLASBUMIN® normal human serum albumin) added up to concentrations of 10%, preferably about 2%. The invention further contemplates the use of any of the above basal media wherein animal-derived proteins are replaced with recombinant human proteins and animal-derived serum, such as BSA, is replaced with human albumin. In preferred embodiments, the media is serum-free in addition to being animal-free.

Additional proliferation—Optionally, other proliferation factors are used. In one embodiment, human recombinant epidermal growth factor (EGF), at a concentration of between 0-1 μg/mL is used. In a preferred embodiment, the EGF concentration is around 10 ng/mL. Alternative growth factors which may be used include, but are not limited to, TGFα or TGFβ2 (5 ng/mL; range 0.1-100 ng/mL), activin A, cholera toxin (preferably at a level of about 0.1 μg/mL; range 0-10 μg/mL), transferrin (5 μg/mL; range 0.1-100 μg/mL), fibroblast growth factors (bFGF 40 ng/mL (range 0-200 ng/mL), aFGF, FGF-4, FGF-8; (all in range 0-200 ng/mL), bone morphogenic proteins (i.e. BMP-4) or other growth factors known to enhance cell proliferation.

Generation of Conditioned Medium

Generation of ACCS—The AMP cells of the invention can be used to generate ACCS. In one embodiment, the AMP cells are isolated as described herein and 1×10⁶ cells/mL are seeded into T75 flasks containing between 5-30 mL culture medium, preferably between 10-25 mL culture medium, and most preferably about 10 mL culture medium. The cells are cultured until confluent, the medium is changed and in one embodiment the ACCS is collected 1 day post-confluence. In another embodiment the medium is changed and ACCS is collected 2 days post-confluence. In another embodiment the medium is changed and ACCS is collected 4 days post-confluence. In another embodiment the medium is changed and ACCS is collected 5 days post-confluence. In a preferred embodiment the medium is changed and ACCS is collected 3 days post-confluence. In another preferred embodiment the medium is changed and ACCS is collected 3, 4, 5, 6 or more days post-confluence. Skilled artisans will recognize that other embodiments for collecting ACCS from AMP cell cultures, such as using other tissue culture vessels, including but not limited to cell factories, flasks, hollow fibers, or suspension culture apparatus, or collecting ACCS from sub-confluent and/or actively proliferating cultures, are also contemplated by the methods of the invention. It is also contemplated by the instant invention that the ACCS be cryopreserved following collection. It is also contemplated by the invention that ACCS be lyophilized following collection. It is also contemplated by the invention that ACCS be formulated for sustained-release following collection. It is also contemplated by the invention that ACCS be formulated for an aerosol following collection. It is also contemplated that ACCS production be scaled up for generation of sufficient product for clinical testing and for commercialization. Skilled artisans are familiar with cryopreservation, lyophilization, sustained-release and aerosol formulation methodologies.

Compositions Comprising Hematopoietic Cell Lineages—

The compositions of the invention can be prepared in a variety of ways depending on the intended use of the compositions. For example, a composition useful in practicing the invention may be a liquid comprising an agent of the invention, i.e. one or more undifferentiated or differentiated populations of cells, or combinations thereof, in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous and muco-adhesive.

Pharmaceutical Compositions—The present invention provides pharmaceutical compositions of undifferentiated or differentiated populations of cells, or combinations thereof, and a pharmaceutically acceptable carrier. The present invention also provides pharmaceutical compositions of blood components (i.e. platelets) and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Treatment Kits—The invention also provides for an article of manufacture comprising packaging material and a pharmaceutical composition of the invention contained within the packaging material, wherein the pharmaceutical composition comprises compositions of undifferentiated or differentiated populations of cells, or combinations thereof. In preferred embodiments, the compositions of differentiated populations of cells are one or more hematopoietic lineage cells. In another preferred embodiment, the compositions of undifferentiated populations of cells are AMP cells, optionally in combination with one or more hematopoietic stem or progenitor cells. The packaging material comprises a label or package insert which indicates that the compositions of cells can be used for blood transfusion, bone marrow transplantation, etc.

Formulation, Dosage and Administration

Compositions comprising undifferentiated or differentiated populations of cells, or combinations thereof, may be administered to a subject to provide various cellular or tissue functions, for example, to reconstitute bone marrow following bone marrow ablation or to transfuse blood following blood loss due to trauma, surgery, etc. As used herein “subject” may mean either a human or non-human animal.

Such compositions may be formulated in any conventional manner using one or more physiologically acceptable carriers optionally comprising excipients and auxiliaries. Proper formulation is dependent upon the route of administration chosen. The compositions may be packaged with written instructions for their use in bone marrow transplantation or blood transfusion or restoring a therapeutically important metabolic function. The compositions may also be administered to the recipient in one or more physiologically acceptable carriers. Carriers for the cells may include but are not limited to solutions of phosphate buffered saline (PBS) or lactated Ringer's solution containing a mixture of salts in physiologic concentrations, basal culture media, and the like.

One of skill in the art may readily determine the appropriate concentration, or dose, of the compositions of undifferentiated or differentiated populations of cells, or combinations thereof, for a particular purpose. The skilled artisan will recognize that a preferred dose is one which produces a therapeutic effect, such as reconstituting bone marrow following bone marrow ablation or increasing blood volume to normal levels following blood loss, in a patient in need thereof. Of course, proper doses of the compositions of undifferentiated or differentiated populations of cells, or combinations thereof will require empirical determination at time of use based on several variables including but not limited to the severity and type of disease, injury, disorder or condition being treated; patient age, weight, sex, health; other medications and treatments being administered to the patient; and the like. One of skill in the art will also recognize that number of doses (dosing regimen) to be administered needs also to be empirically determined based on, for example, severity and type of disease, injury, disorder or condition being treated. In a preferred embodiment, one dose is sufficient. Other preferred embodiments contemplate, 2, 3, 4, or more doses

Skilled artisans will recognize that any and all of the standard methods and modalities for bone marrow transplantation, blood transfusion and therapeutic use of blood components currently in clinical practice and clinical development are suitable for using the compositions and practicing the methods of the invention. The compositions of the invention can be administered by injection into a target site of a subject, preferably via a delivery device, such as a tube, e.g., catheter. In a preferred embodiment, the tube additionally contains a needle, e.g., a syringe, through which the compositions can be introduced into the subject at a desired location. Specific, non-limiting examples of administering cells to subjects may also include administration by subcutaneous injection, intramuscular injection, intravenous injection, intraarterial intramuscular, intracardiac injection, infusion, intradermal injection, intrathecal injection, epidural injection, intraperitoneal injection, or intracerebral injection. If administration is intravenous, an injectable liquid suspension of the compositions can be prepared and administered by a continuous drip or as a bolus.

It may be desirable to add other agents, including active agents and/or inactive agents to the stem cell/AMP cell co-cultures or stem cell/AMP cell conditioned medium (including ACCS) cultures. Active agents include but are not limited to growth factors, cytokines, chemokines, antibodies, antibiotics, anti-fungals, anti-virals, small molecules, inhibitors, other cell types, and the like. Inactive agents include carriers, diluents, stabilizers, gelling agents, delivery vehicles, ECMs (natural and synthetic), scaffolds, and the like.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of AMP Cell Compositions

Recovery of AMP cells—Amnion epithelial cells were dissociated from starting amniotic membrane using the dissociation agent PXXIII. The average weight range of an amnion was 18-27 g. The number of cells recovered per g of amnion was about 10-15×10⁶.

Method of selecting AMP cells: Amnion epithelial cells were plated immediately upon isolation from the amnion. After ˜2-3 days in culture, non-adherent cells were removed and the adherent cells were kept. The adherent cells represent about 30% of the plated cells. This attachment to a plastic tissue culture vessel is the selection method used to obtain the desired population of AMP cells. Adherent and non-adherent cells appear to have similar cell surface marker expression profiles but the adherent cells have greater viability and are the desired population of cells. Selected AMP cells were cultured until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2

### Generation of ACCS

The AMP cells of the invention can be used to generate ACCS. The AMP cells were isolated as described herein and 1×10⁶/mL cells were seeded into T75 flasks containing 10 mL culture medium. The cells are cultured until confluent, the medium is changed and ACCS was collected 3 days post-confluence. Other collection time points are contemplated by the methods of the invention as well. Skilled artisans will recognize that other embodiments for collecting ACCS from confluent cultures, such as using other tissue culture vessels, including but not limited to cell factories, flasks, hollow fibers, or suspension culture apparatus, are also contemplated by the methods of the invention. It is also contemplated by the instant invention that the ACCS be cryopreserved following collection. It is also contemplated by the invention that ACCS be lyophilized following collection. It is also contemplated by the invention that ACCS be formulated for sustained-release following collection. It is also contemplated by the invention that ACCS be formulated for an aerosol following collection. It is also contemplated that ACCS production be scaled up for generation of sufficient product for clinical testing and for commercialization. Skilled artisans are familiar with cryopreservation, lyophilization, sustained-release and aerosol formulation methodologies.

### Example 3

### Detection of Cytokines Known to Induce Hematopoietic Differentiation

To determine which cytokines known to induce hematopoietic differentiation may be secreted by the AMP cells of the present invention, ACCS was isolated from cell cultures that were seeded onto tissue culture treated flasks at a density of 40,000 cells per cm². Cells were cultured in a proprietary serum-free medium supplemented with 10 ng/mL of EGF. Culture media was exchanged every 2 days during the growth period. After cells reached near confluency (˜1-2 wk after isolation), fresh media was applied and ACCS was collected after three days and stored at −80° C. for subsequent analysis.

ELISAs were performed on conditioned media (ACCS) derived from AMP cells obtained from 10 different placentas (non-pooled ACCS). In addition to assaying each ACCS sample individually, pooled ACCS samples were also tested to determine if variability of ELISA results between samples could be reduced. ACCS was obtained as described above. Pool 1 was comprised of ACCS from placentas 1-5, Pool 2 was comprised of ACCS from placentas 6-10, and Pool 3 was comprised of ACCS from placentas 1-10. Results: VEGF was expressed at the physiological level of ˜5-16 ng/mL.

### Example 4

### Differentiation of Stem Cells into Hematopoietic Cell Lineages Using Amp Cell Feeder Layers, Co-Cultures and/or ACCS

AMP cells are collected as described above. The AMP cells are used to create feeder layers for stem cells as described in, for example, Miyamoto, K., et al., Stem Cells 2004; 22:433-440, or used in standard co-cultures. Once feeder layers are established, stem cells (embryonic stem cells, fetal stem cells, extraembryonic stem cells or adult stem cells (i.e. hematopoietic stem cells, muscle stem cells, adipose stem cells) are plated as described, for example, in Miyamoto, K., et al., Stem Cells 2004; 22:433-440. After culture, the stem Cells are tested for the expression of hematopoietic stem cell markers such as CD34, CD45, CD31, CD38 and glycophorin A.

In a variation of the feeder-layer and co-culture experiments described above, the culture media is supplemented with ACCS. In this experiment, the culture media is supplemented with 10%, 20%, 30%, 40%, 50%, 60%, 70%, 80%, and 90% ACCS. In one aspect of the experiment, the culture media is replaced with 100% ACCS. After culture, the stem cells are tested for the expression of hematopoietic stem cell markers such as CD34, CD45, CD31, CD38 and glycophorin A.

In another experiment, the stem cells (embryonic stem cells, fetal stem cells, extraembryonic stem cells or adult stem cells (i.e. hematopoietic stem cells, muscle stem cells, adipose stem cells) are plated and then cultured in ACCS in the absence of an AMP cell feeder-layer or co-culture. After culture, the stem cells are tested for the expression of hematopoietic stem cell markers such as CD34, CD45, CD31, CD38 and glycophorin A.

### Example 5

### Differentiation of AMP Cells into Hematopoietic Cell Lineages

Using standard hematopoietic differentiation protocols (see, for example, Tian, X., et al., 2004, Exp Hemat 32:1000-1009; Orkin, S. H., et al., 2000, Nat Rev Genet 1:57-64; Helgason C. D., et al., 1996 Blood 87:2740-2749; Wiles M. V. & Keller, G., 1991, Development (Cambridge, U.K.) 111:259-267), AMP cells are differentiated into hematopoietic cell lineages. To assess whether the AMP cells have been differentiated, the cells are tested for the expression of hematopoietic stem cell markers such as CD34, CD45, CD31, CD38 and glycophorin A.

### Example 6

### Use of AMP Cells to Increase Differentiation Efficiency of Hematopoietic Stem/Progenitor

Preliminary experiments evaluated the ability of AMP cells to support the ex vivo differentiation efficiency of hematopoietic stem cells (HSC) and hematopoietic progenitor cells (HPC) in comparison with stromal cell-free, cytokine-supplemented cultures. After 7 days, no significant differences in the total nonadherent cell yield in AMP cell co-cultures (89.36-fold) versus cytokine liquid cultures (92.7-fold). In contrast, the frequency of both total CD34+ (24.6% vs. 16.3%) and the more primitive CD34+CD38− (HSC, 21.6% vs. 5.7%) subset was markedly higher in AMP cell co-cultures versus cytokine liquid cultures. These results demonstrate that AMP cells may have a hematopoietic role in vivo, specifically at the more primitive HSC level of development and self-renewal (CD34+CD38− in humans). Furthermore, due to their lack of antigenic surface markers, AMP cells may be useful for transplantation into any patient.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is:

1. A method for differentiating stem cells into hematopoietic lineage cells comprising culturing the stem cells with Amnion-derived Multipotent Progenitor (AMP) cells or Amnion-derived Cellular Cytokine Solution (ACCS).

2. A method for increasing the differentiation efficiency of stem cells in a population comprising culturing the stem cells with Amnion-derived Multipotent Progenitor (AMP) cells or Amnion-derived Cellular Cytokine Solution (ACCS).

3. The method of claim 1 or 2 wherein the AMP cells are a feeder layer in the culture or wherein the AMP cells are co-cultured with the stem cells in the culture.

4. The method of claim 1 or 2 further optionally comprising adding ACCS as a supplement to the stem cell culture.

5. The method of claim 1 or 2 wherein the stem cells are totipotent, pluripotent or multipotent stem cells.

6. The method of claim 5 wherein the stem cells are embryonic stem cells, fetal stem cells, extraembryonic stem cells or adult stem cells.

7. The method of claim 6 wherein the adult stem cells are hematopoietic stem cells or hematopoietic progenitor cells.

8. The method of claim 1 wherein the hematopoietic lineage cells are myeloid and/or lymphoid lineage cells.

9. The method of claim 1 wherein the stem cells are cultured with ACCS prior to differentiation.

10. The method of claim 1 wherein the stem cells are cultured with ACCS during differentiation.

11. A composition of hematopoietic lineage cells made by the method of claim 1.

12. The composition of claim 11 wherein the hematopoietic lineage cells are myeloid lineage cells.

13. The composition of claim 11 wherein the hematopoietic lineage cells are lymphoid lineage cells.

14. The composition of claim 11 which is a pharmaceutical composition.

15. A method of treating a disease or disorder of the hematopoietic system of a patient in need thereof comprising administering to the patient a therapeutic amount of the composition of claim 14.

16. The method of claim 15 wherein the disease or disorder in the hematopoietic system is the result of radiation, chemotherapy, infection, trauma or disease.

17. A kit comprising the pharmaceutical composition of claim 14.
